(19) United States
(12) Patent Application Publication
(10) Pub. No.: US 2009/0105773 A1
(43) Pub. Date: Apr. 23, 2009

(54) METHOD AND APPARATUS FOR INSERTION OF AN INTERSPINOUS PROCESS DEVICE

(75) Inventors: Eric C. LANGE, Collierville, TN (US); Kidong YU, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 11/877,422

(22) Filed: Oct. 23, 2007

(51) Int. Cl. A61F 5/00; A61B 17/70; A61B 19/00

(52) U.S. Cl. 606/86 A; 128/898; 606/249

(57) ABSTRACT

A method of inserting an implant between adjacent interspinous processes that includes, in one embodiment, providing an implant that is configurable to a collapsed configuration and to an expanded configuration, collapsing the implant, providing the collapsed implant in a positioning device, positioning a distal end of the positioning device in a patient such that the distal end is located between a pair of adjacent interspinous processes, and pushing the collapsed implant through the positioning device while removing the positioning device from the patient such that the implant is inserted between and adjacent to the interspinous processes to hold the adjacent vertebrae in a corrected position.

The present disclosure relates to devices used in orthopedic surgical procedures and methods of inserting those devices in a medical patient. Specifically, the present disclosure relates to devices that can be used to correctly position a pair of adjacent interspinous processes and methods of inserting these devices between the pair of adjacent interspinous processes.

Surgical techniques have been developed to treat spinal stenosis, a condition of the spine characterized by a narrowing of the spinal canal. With spinal stenosis, the spinal canal narrows and pinches the spinal cord and nerves, causing pain in the back and legs. One such surgical technique developed is to separate a pair of adjacent vertebrae and insert an interspinous implant between the interspinous processes to maintain the desired separation between the vertebrae. However, the steps required to separate the pair of adjacent vertebrae and to insert the interspinous implant can be time consuming and difficult since different instruments are often used to perform each step.

As with any surgery, one consideration when performing surgery to insert an interspinous implant between adjacent vertebrae is the size of the incision that is required to allow introduction of the implant. Minimally invasive techniques are generally preferred since the patient usually requires less recovery time than with a traditional or open surgery. For a minimally invasive surgery, a small incision in the patient is created to form an implantation profile in which instruments and an interspinous implant are inserted into a patient. Next, the surgeon using the instruments must carefully separate the pair of adjacent vertebrae and insert the interspinous implant between the interspinous processes on the pair of vertebrae.

Working through a small incision to insert instruments and an interspinous implant between a pair of vertebrae requires particular devices as well as abundant care on the part of the surgeon.

### BRIEF DESCRIPTION OF THE DRAWINGS

FIG. 1 is a perspective view of one embodiment of an interspinous implant being implanted between a pair of interspinous processes.

FIG. 2 is a perspective view of the FIG. 1 interspinous implant in a collapsed configuration.

FIG. 3 is a perspective view of the embodiment of the FIG. 1 interspinous implant being implanted between a pair of interspinous processes.

FIG. 4 is a perspective view of the embodiment of the FIG. 1 interspinous implant implanted between a pair of interspinous processes.

FIG. 5 is a perspective view of the FIG. 4 interspinous implant in an expanded configuration.

FIG. 6 is a perspective view of another embodiment of an interspinous implant being implanted between a pair of interspinous processes.

FIG. 7 is a perspective view of the embodiment of the FIG. 6 interspinous implant being implanted between a pair of interspinous processes.

FIG. 8 is a perspective view of the embodiment of the FIG. 6 interspinous implant being implanted between a pair of interspinous processes.

FIG. 9 is a perspective view of the embodiment of the FIG. 6 interspinous implant implanted between a pair of interspinous processes.

FIG. 10 is a perspective view of one embodiment of a positioning instrument between a pair of interspinous processes in a medical patient.

FIG. 11 is a perspective view of another embodiment of a positioning instrument between a pair of interspinous processes in a medical patient.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated devices, and such further applications of the principles of the disclosure as illustrated therein, being contemplated as would normally occur to one skilled in the art to which the disclosure relates.

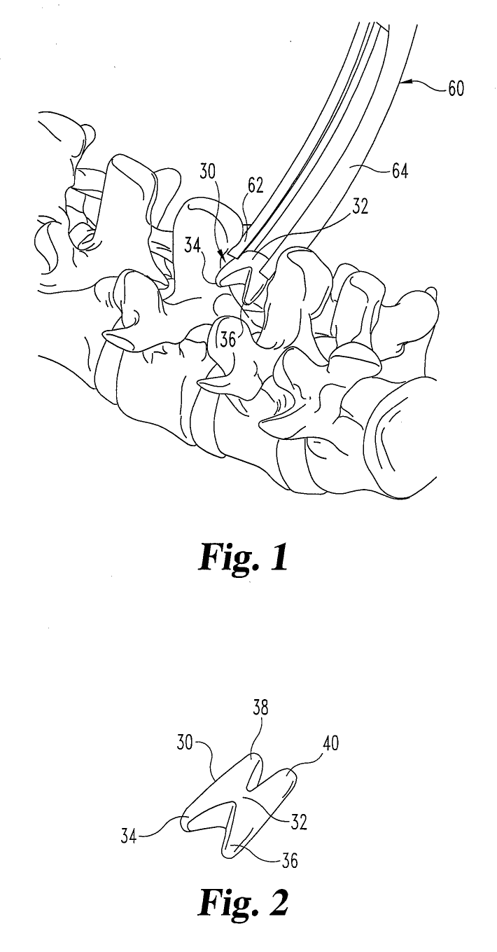

Referring generally to FIGS. 1, 2, 3, 4, and 5, there are shown embodiments of an implant 30. In the illustrated embodiment, implant 30 includes a body portion 32, a leg 34, a leg 36, a leg 38, and a leg 40. As illustrated, legs 34, 36, 38, and 40 are all identical. However, in other embodiments, legs 34, 36, 38, and 40 can be dissimilar from each other. For example, in other embodiments, legs 34, 36, 38, and 40 can be different sizes and/or shapes. As discussed further below, implant 30 can be connected to bone or other tissue, for example, between a pair of adjacent interspinous processes of the adjacent vertebrae, and can be used in connection with a variety of orthopedic devices, such as orthopedic plates, connectors, rods, fusion cages, disc prostheses, or other types of instrumentation or implants.

Implant 30 is formed of a flexible material which allows legs 34, 36, 38, and 40 to bend during insertion of implant 30 between a pair of interspinous processes and to flex back to implant 30's original shape after insertion. As illustrated in FIG. 5, implant 30 forms an “H”-shaped configuration in an expanded configuration, and in FIG. 2, implant 30 forms an “X”-shaped configuration when in a collapsed configuration.

As illustrated, body portion 32 has a thickness slightly greater than legs 34, 36, 38, and 40. In other forms, body portion 32 has a thickness equal to legs 34, 36, 38, and 40. Body portion 32 includes a pair of curved surfaces 33 to engage the interspinous processes. As illustrated, legs 34, 36, 38, and 40 each form a triangular or wedge shape; however, in other forms, legs 34, 36, 38, and 40 can be shaped differently. By non-limiting example, legs 34, 36, 38, and 40 can be rectangle or curved shapes.

Implant 30 may be formed from a wide variety of biocompatible materials that can undergo reversible elastic deformation. Examples of such materials include elastic or rubbery polymers, hydrogels, or other hydrophilic polymers, or composites thereof. Some suitable elastomers include silicone, polyurethane, copolymers of silicone and polyurethane, polyolefins, neoprene, nitrile, vulcanized rubber, and combinations thereof. In other embodiments, implant 30 is made of a metal that can undergo reversible elastic deformation, such as shape-memory metals or nickel-titanium.

The nature of the materials employed to form implant 30 should be selected so that implant 30 has sufficient load-bearing capacity. For example, in some embodiments, a compressive modulus of at least about 0.1 Mpa is desired, although compressive strengths in the range of about 1 Mpa to about 20 Mpa are also desired. Often the compressive modulus is at least about 5 Mpa.

In some embodiments, implant 30 may also deliver desired pharmacological agents. The pharmacological agent may be a growth factor that may repair damaged tissue or bone and may include an osteoinductive factor, transforming growth factors, a platelet-derived growth factor, or other similar growth factors or combinations thereof having the ability to repair tissue or bone.

In other forms, implant 30 may comprise a pharmacological agent used for treating various spinal conditions, including degenerative disc disease, spinal arthritis, spinal infection, spinal tumor, and osteoporosis. Such agents include antibiotics, analgesics, anti-inflammatory drugs, including steroids, and combinations thereof.

The pharmacological agents, if any, can be dispersed within implant 30 for in vivo release. The pharmacological agents may be dispersed in implant 30 by adding the agents to implant 30 when it is formed, by soaking a formed implant 30 in an appropriate solution containing the agent, or by other appropriate methods. In other forms, the pharmacological agents may be chemically or otherwise associated with implant 30. For example, the agents may be chemically attached to an outer surface of implant 30.

In some embodiments, implant 30 may include an x-ray marker, such as a tantalum marker, to assist in positioning the implant. In other embodiments, a combination of larger x-ray markers and smaller x-ray markers may be used to facilitate observing the orientation of implant 30 when it is implanted into a medical patient. The x-ray markers can be more readily observed on x-rays, making the positioning and orientation of implant 30 more easily observed and corrected. To use implant 30, legs 34, 36, 38, and 40 are manipulated to form an “X”-shaped configuration with body portion 32 as shown in FIG. 1. This “X”-shaped configuration enables implant 30 to be inserted between a pair of adjacent interspinous processes. This manipulation converts an “H”-shaped implant 30 to an “X”-shaped implant by folding the upwardly and downwardly extending legs so that they extend substantially horizontally in a direction that is generally parallel to the body portion 32 of the “H”-shaped implant. When implant 30 is manipulated to its collapsed/implantable configuration, the implantation profile of implant 30 is reduced. The reduced profile of implant 30 assists a surgeon by allowing the surgeon to form a smaller incision and perform a minimally-invasive procedure.

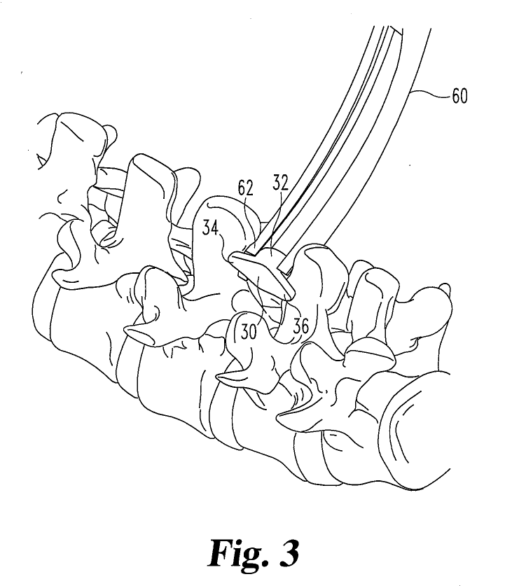

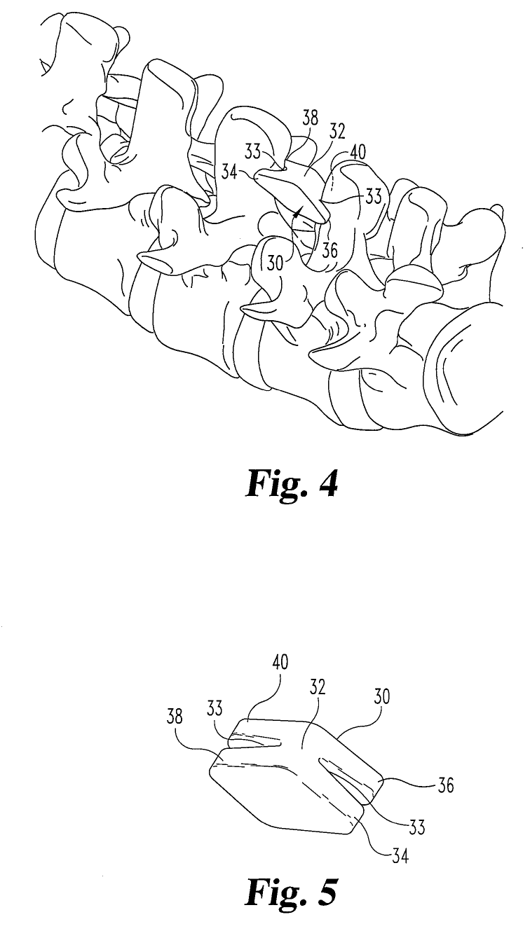

Also shown in FIGS. 1, 2, 3, 4, and 5 is an embodiment of a method for implanting implant 30 in a medical patient. In FIG. 1, implant 30 is loaded into a positioning instrument 60 while implant 30 is in its collapsed/implantable configuration. Positioning instrument 60 is positioned between two interspinous processes, with a distal portion 62 of the positioning instrument 60 extending just beyond the interspinous processes when a ramp 64 is inserted. When ramp 64 is positioned, implant 30 is pushed down ramp 64 such that the leading pair of legs 34, 36 begin to unfold from the collapsed/implantable configuration to start to form the expanded/relaxed configuration as shown in FIG. 3. As the legs unfold, they extend upward and downward along one side of two interspinous processes as shown in FIGS. 3 and 4. Ramp 64 is then withdrawn as implant 30 is ejected as shown in FIGS. 3 and 4. Body portion 32 is positioned between the two interspinous processes, and the second pair of legs 38, 40 unfold to extend upward and downward along the second side of the interspinous processes as shown in FIG. 4. FIG. 4 shows an implant 30 after implantation in a medical patient. Legs 34, 36, 38, and 40 of implant 30 grip the interspinous processes to hold the implant 30 in position.

In other embodiments, implant 30 can have indents and/or other surface features to facilitate collapsing and implanting implant 30 or to avoid cracking or tearing implant 30 when legs 34, 36, 38, and 40 are folded and unfolded. Features such as ridges to facilitate gripping the interspinous processes may also be included on implant 30.

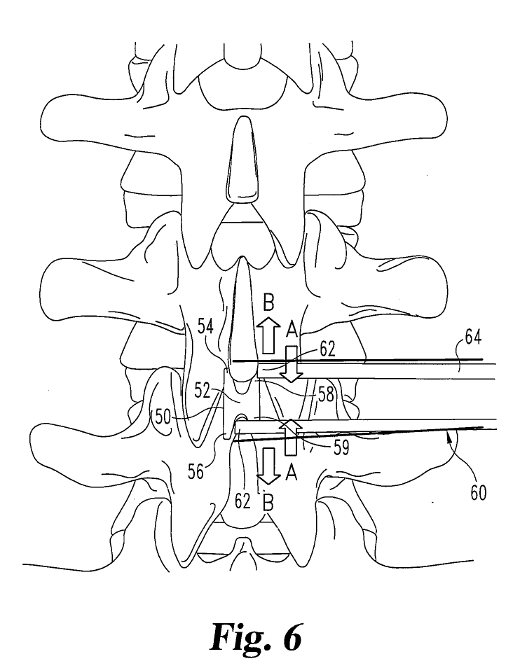

Referring generally to FIGS. 6, 7, 8, and 9, there is shown an implant 50. Implant 50 is similar to implant 30. Implant 50 includes a body portion 52 and four legs 54, 56, 58, and 59 extending from body portion 52. However, implant 50 is substantially rigid, whereas implant 30 is flexible.

Implant 50 may be formed from a wide variety of biocompatible materials that are substantially rigid. Examples of such materials include plastics, metal, and/or combinations thereof. The nature of materials selected to form implant 50 should be selected such that implant 50 has a sufficient load-bearing capacity. In preferred embodiments, a compressive modulus of at least about 0.1 Mpa is desired, although compressive strengths in the range of about 1 Mpa to about 20 Mpa are more preferred. In other embodiments, the compressive modulus is at least about 5 Mpa.

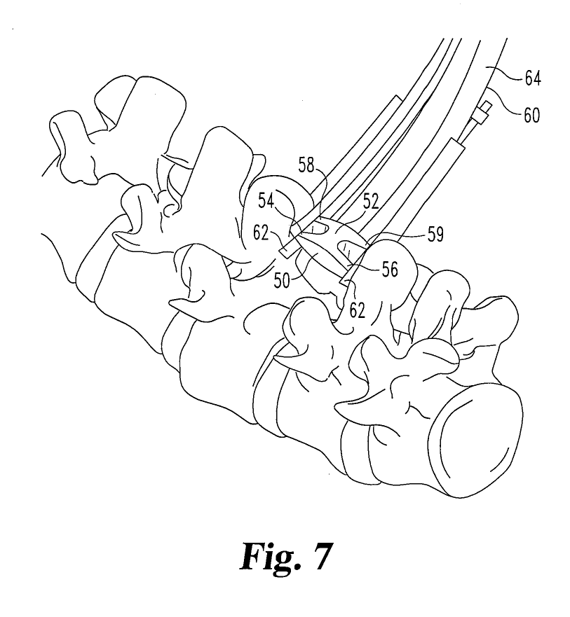

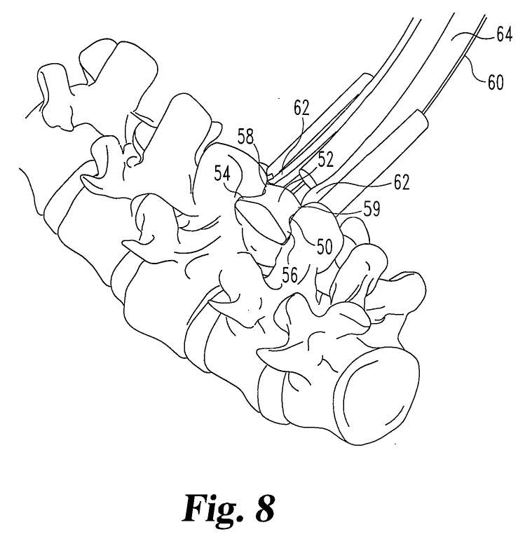

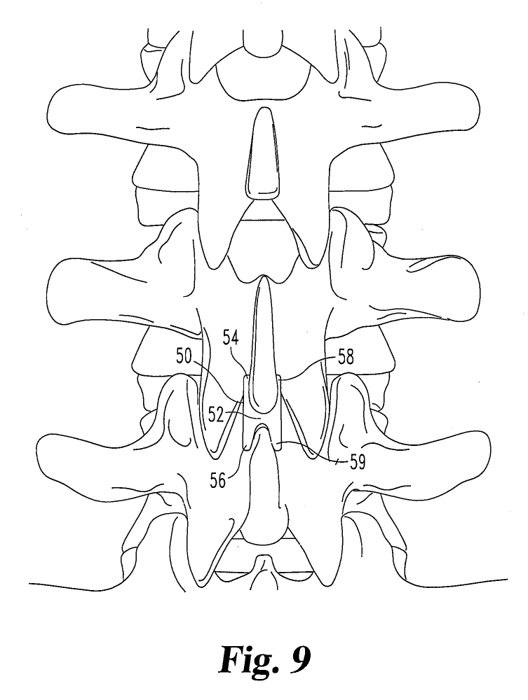

To use implant 50, implant 50 is inserted in ramp 64 of positioning instrument 60. Implant 50 is then moved or pushed along ramp 64 to distal portion 62. As illustrated by pair of arrows A in FIG. 6, distal portion 62 is positioned between a pair of adjacent interspinous processes of a pair of vertebrae. As shown, one leg of distal portion 62 is offset from the other leg of distal portion 62 to allow legs 54, 56 to exit ramp 64. Immediately before legs 54, 56 exit ramp 64, both legs of distal portion 62 come into full decompression until the legs 54, 56 of implant 50 exit from the end of distal portion 62 as shown in FIG. 6. Illustrated in FIG. 7 is full decompression of the legs of distal portion 62. In FIG. 8, a view of legs 54, 56 of implant 50 snapping out of distal portion 62 of ramp 64 is illustrated. In FIG. 6, pair of arrows B represent the motion of the legs of distal portion 62 as implant 50 is snapping out of distal portion 62. Illustrated in FIG. 9 is the final position of implant 50 after it has been inserted between a pair of adjacent interspinous processes. Distal portion 62 of ramp 64 has been pulled back or removed from the medical patient while implant 50 continues to exit from distal portion 62. FIG. 9 illustrates implant 50 in its final position with legs 54, 56, 58, and 59 resting longitudinally along the pair of adjacent interspinous processes. After insertion of implant 50, the adjacent pair of interspinous processes go into compression as determined by the body portion 52 of implant 50 contacting and separating the interspinous processes.

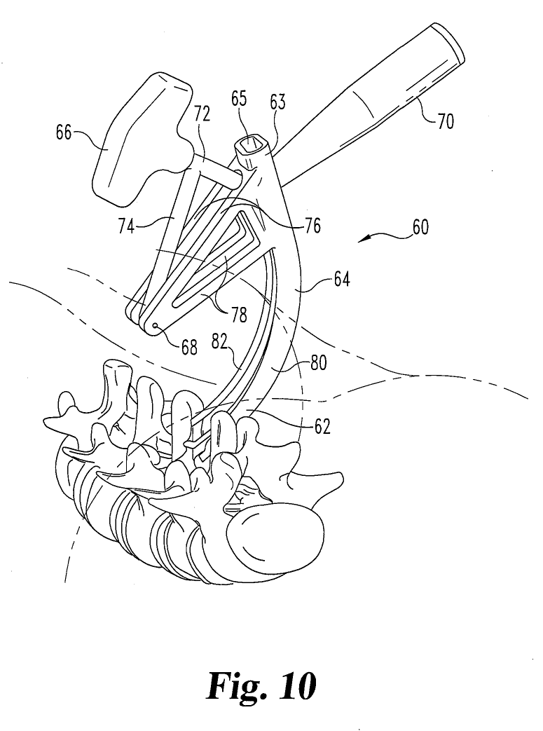

FIG. 10 illustrates one embodiment of positioning instrument 60. As described above, positioning instrument 60 includes distal portion 62 and ramp 64. In this embodiment, positioning instrument 60 also includes a pusher handle 66, a pusher pivot 68, a ramp-secure handle 70, a pusher 72, a pusher arm 74, ramp upper arms 76, and ramp lower arms 78. Positioning instrument 60 also includes a proximal portion 63 opposite distal portion 62 and an implant entry 65 near proximal portion 63. Ramp 64 includes a first ramp arm 80 and a second ramp arm 82. In the illustrated embodiment, first ramp arm 80 and second ramp arm 82 form a “C” shape; however, in other embodiments, first ramp arm 80 and second ramp arm 82 may form another shape or may be straight. In the illustrated embodiment, pusher handle 66 is ergonomically shaped to receive a hand of the surgeon using the positioning instrument 60. Additionally, ramp secure handle 70 is configured to receive the other hand of the surgeon using the positioning instrument 60. Pusher 72 is configured to slide within ramp 64 as the surgeon pushes down on pusher handle 66 or pulls up on pusher handle 66. Additionally, pusher 72 is configured to contact and move an implant inserted in implant entry 65. Pusher arm 74 is substantially straight between pusher handle 66 and pusher pivot 68. Further, pusher arm 74 is configured to rotate about pusher pivot 68 as the surgeon pushes down or pulls up pusher handle 66 to slide pusher 72 in ramp 64. Ramp upper arms 76 and ramp lower arms 78 extend from ramp 64 to join at pusher pivot 68. In the illustrated embodiment, ramp upper arms 76, ramp lower arms 78, and ramp 64 form a triangle; however, in other embodiments, ramp upper arms 76 and ramp lower arms 78 may be configured differently.

To use positioning instrument 60, a surgeon forms an incision in the medical patient. Next, the surgeon inserts the distal portion 62 of ramp 64 through the incision in the medical patient and positions the distal portion 62 between a pair of adjacent interspinous processes. Next, the surgeon loads an implant, such as implant 30 or implant 50, in the implant entry 65. The surgeon then pushes down on pusher handle 66 while holding ramp secure handle 70 to steady the positioning instrument 60. Next, the surgeon continues to push pusher handle 66 down thereby moving pusher 72 into ramp 64 to engage the implant and move the implant along the ramp 64. As the first pair of legs of the implant exit the distal portion 62, a surgeon starts to remove distal portion 62 from between the interspinous processes. The first pair of legs of the implant are positioned longitudinally along one side of the interspinous processes. By removing distal portion 62 from the interspinous processes and pushing pusher handle 66 towards ramp 64, the remaining two legs of the implant exit from distal portion 62 and are positioned longitudinally along the other side of the interspinous processes. During this process, all four legs of the implant are positioned longitudinally along the adjacent pair of interspinous processes. The adjacent pair of vertebrae are now separated to a corrected position by the implant. The implant positioned between the interspinous processes can be further connected to other orthopedic devices.

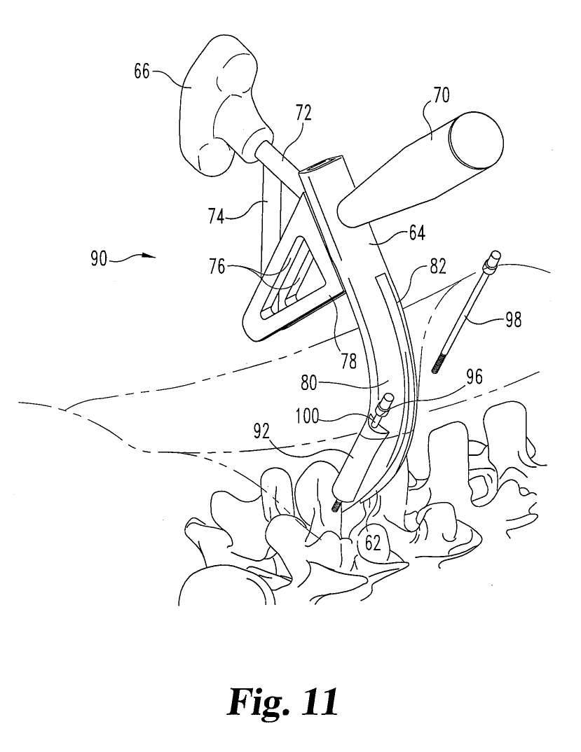

Illustrated in FIG. 11 is an embodiment of a positioning instrument 90. Positioning instrument 90 is similar to positioning instrument 60; however, positioning instrument 90 includes a first anchor holder 92 and a second anchor holder 94 (not shown in FIG. 11). First anchor holder 92 and second anchor holder 94 are configured to receive a first anchor 96 and a second anchor 98. First anchor holder 92 is a half-cylindrical shape defining a hole 100 for receiving first anchor 96. First hole 100 is sized to snugly receive first anchor 96. Likewise, although not shown in FIG. 11, second anchor holder 94 is also a half-cylindrical shape with a second hole 102 for receiving second anchor 98. Likewise, second hole 102 is sized to snugly receive second anchor 98. In the illustrated embodiment in FIG. 11, first anchor 96 and second anchor 98 each have an elongated shaft with a first end being threaded and a second end having a cap for receiving an instrument to insert first anchor 96 or second anchor 98 into either first hole 100 or second hole 102. The threaded end of first anchor 96 or second anchor 98 is screwed or twisted into a portion of the vertebrae in which the implant is to be inserted between. The first anchor 96 or second anchor 98 can be used to stabilize the positioning instrument 90 for insertion of the implant between corresponding interspinous processes.

While the disclosure has been illustrated and described in detail in the drawings and foregoing description, the same is to be considered as illustrative and not restrictive in character, it being understood that only the preferred embodiment has been shown and described and that all changes and modifications that come within the spirit of the disclosure are desired to be protected.

What is claimed is:

1. A method of inserting an interspinous implant between adjacent interspinous processes comprising:
providing an implant that is configurable to a collapsed configuration and to an expanded configuration, wherein the implant has at least two pairs of legs that form an “X”-shaped configuration when in the collapsed configuration and said pairs of legs form an “H”-shaped configuration when in the expanded configuration;
causing the implant to assume its collapsed configuration;
providing said collapsed implant in a positioning device for holding said implant in its collapsed configuration to facilitate implantation in a patient, said positioning device having a proximal end and a distal end;
positioning the distal end of the positioning device in a patient such that the distal end of the positioning device is located between a pair of adjacent interspinous processes;
pushing the collapsed implant through the positioning device until one pair of the legs exit the distal end of the device and position themselves longitudinally beside the adjacent interspinous processes; and
removing the positioning device from the patient while pushing the partially collapsed implant through the positioning device until the other pair of legs exit the distal end of the positioning device and position themselves longitudinally on the other side of the adjacent interspinous processes.

2. The method of claim 1, further comprising connecting said implant to at least one other orthopedic implant.

3. The method of claim 1, further comprising connecting said implant to at least one orthopedic device.

4. The method of claim 1, further comprising bending said implant so that said implant closely grips the vertebrae.

5. The method of claim 1, wherein said implant includes a body portion having a pair of substantially concave surfaces, and said pushing the collapsed implant includes positioning each concave surface adjacent an interspinous process.

6. The method of claim 1, further comprising anchoring the positioning device to the interspinous processes.

7. A method of inserting an interspinous implant between adjacent interspinous processes comprising:
providing a substantially rigid implant, wherein the implant has at least two pairs of legs that form an “H”-shaped configuration;
providing said implant in a positioning device for holding said implant to facilitate implantation in a patient, said positioning device having a first ramp arm and a second ramp arm, each of said ramp arms having a proximal end and a distal end;
positioning the distal ends of the ramp arms in a patient such that the distal ends of the ramp arms are located between a pair of adjacent interspinous processes;
pushing the implant along the length of the first ramp arm and the second ramp arm to spread the ramp arms apart until one pair of the legs exits the distal ends of the ramp arms and position themselves longitudinally beside the adjacent interspinous processes; and
removing the device from the patient while pushing the implant along the length of the ramp arms to spread the ramp arms apart until the other pair of legs exit the distal ends of the ramp arms and position themselves longitudinally on the other side of the adjacent interspinous processes.

8. The method of claim 7, further comprising connecting said implant to at least one other orthopedic implant.

9. The method of claim 7, further comprising connecting said implant to at least one orthopedic device.

10. The method of claim 7, wherein said implant includes a body portion having a first concave surface and a second concave surface and said pushing the implant includes positioning each concave surface against one of the interspinous processes.

11. A method for decompressing a pair of interspinous processes, comprising:
placing a positioning device between a pair of adjacent interspinous processes, said positioning device including a ramp, a pusher, and defining an implant entry, said ramp configured to receive said pusher;
securing a first anchor to said ramp and a first vertebra;
securing a second anchor to said ramp and a second vertebra;
inserting an implant in said implant entry;
moving said pusher in said ramp to engage said implant and to push said implant along said ramp; and
inserting said implant between said first interspinous process and said second interspinous process.

12. The method of claim 11, wherein said positioning device includes a ramp secure handle, and said moving act includes holding said ramp secure handle to stabilize said ramp.

13. The method of claim 11, wherein said positioning device includes a pusher pivot, and said moving act includes rotating said pusher about said pusher pivot.

14. The method of claim 11 wherein said moving act includes sliding said pusher in said ramp.

15. The method of claim 11, wherein said implant includes at least two pairs of legs that form an “X”-shaped configuration when in a collapsed configuration and said pairs of legs form an “H”-shaped configuration when in an expanded configuration, said inserting said implant between said first interspinous process and said second interspinous process act includes pushing a collapsed implant along the ramp until one pair of the legs exit the ramp and position themselves longitudinally beside the adjacent interspinous processes, and removing the ramp from the patient while pushing the partially collapsed implant along the ramp until the other pair of legs exit the ramp and position themselves longitudinally on the other side of the adjacent interspinous processes.

16. The method of claim 11 wherein said ramp includes a pair of ramp arms, each of said pair of ramp arms having a proximal end and a distal end, and said implant includes at least two pairs of legs that form an “H”-shaped configuration, and said placing act includes positioning the distal ends of the ramp arms in a patient such that the distal ends of the ramp arms are located between a pair of adjacent interspinous processes, said inserting said implant between said first interspinous process and said second interspinous process includes pushing the implant along the length of the ramp arms to spread the ramp arms apart until one pair of the legs exit the distal ends of the ramp arms and position themselves longitudinally beside the adjacent interspinous processes; and
removing the positioning device from the patient while pushing the implant along the length of the ramp arms to spread the ramp arms apart until the other pair of legs exit the distal ends of the ramp arms and position themselves longitudinally on the other side of the adjacent interspinous processes.

17. A spinal positioning apparatus, comprising:
an elongate ramp including a distal portion and a proximal portion, said distal portion sized to fit between a pair of adjacent interspinous processes, said proximal portion defining an implant entry sized to receive an implant;
a pusher pivot attached to said ramp;
a pusher configured to slide within said ramp and to engage said implant, said pusher configured to rotate about said pusher pivot;
a pusher handle attached to said pusher and configured to move said pusher; and
a ramp secure handle attached to said proximal portion of said ramp and configured to stabilize said ramp.

18. The apparatus of claim 17, further comprising a pair of ramp upper arms and a pair of ramp lower arms, both of said pair of arms being connected to said pusher pivot and said ramp.

19. The apparatus of claim 17, further comprising a pusher arm connected to said pusher pivot and said pusher.

20. The apparatus of claim 17, wherein said elongate ramp is curved between said distal portion and said proximal portion.

21. The apparatus of claim 17, further comprising:
a first anchor holder positioned near said distal portion of said ramp; and
a second anchor holder positioned near said distal portion of said ramp.

22. The apparatus of claim 21, wherein said first anchor holder defines a first hole sized to receive a first anchor and said second anchor holder defines a second hole sized to receive a second anchor.
